(19) United States
(12) Patent Application Publication
(10) Pub. No.: US 2012/0083678 A1
(43) Pub. Date: Apr. 5, 2012

(54) System and method for raman chemical analysis of lung cancer with digital staining

(75) Inventors: Amy Drauch, Carnegie, PA (US); John Maier, Pittsburgh, PA (US)

(73) Assignee: ChemImage Corporation, Pittsburgh, PA (US)

(21) Appl. No.: 13/200,779

(22) Filed: Sep. 30, 2011

Related U.S. Application Data
(60) Provisional application No. 61/404,265, filed on Sep. 30, 2010.

(51) Int. Cl. A61B 5/1455; G06F 19/00; G01J 3/44

(52) U.S. Cl. 600/310; 356/301; 702/19

(57) ABSTRACT

The present disclosure provides for a system and method for diagnosing biological samples that combines the visual staining features familiar to pathologists with the accurate, reliable, and nondestructive capabilities of Raman chemical imaging. The invention disclosed herein may be applied to diagnose lung cancer samples. A method may comprise illuminating a biological sample to generate interacted photons, filtering said interacted photons using a tunable filter, and detecting interacted photons to generate a test Raman data set representative of said sample. The method may further comprise applying at least one chemometric technique and/or a digital stain to said test Raman data set. This test Raman data set may be analyzed to diagnose said sample as comprising at least one of: adenocarcinoma, mesothelioma, and combinations thereof. A system may comprise an illumination source, a tunable filter, and a detector configured to generate a test Raman data set representative of a biological sample.

### RELATED APPLICATIONS

This Application claims priority under 35 U.S.C. §119(e) to U.S. Provisional Patent Application No. 61/404,265, filed on Sep. 30, 2010, entitled “System And Method For Raman Chemical Analysis Of Lung Cancer,” which is hereby incorporated by reference in its entirety.

### BACKGROUND

The biochemical composition of a cell is a complex mix of biological molecules including, but not limited to, proteins, nucleic acids, lipids, and carbohydrates. The composition and interaction of the biological molecules determines the metabolic state of a cell. The metabolic state of the cell will dictate the type of cell and its function (i.e., red blood cell, epithelial cell, etc.). Tissue is generally understood to mean a group of cells that work together to perform a function. Spectroscopic techniques provide information about the biological molecules contained in cells and tissues and therefore provide information about the metabolic state. As the cell's or tissue's metabolic state changes from the normal state to a diseased state, spectroscopic techniques can provide information to indicate the metabolic change and therefore serve to diagnose and predict the outcome of a disease. Cancer is a prevalent disease, so physicians are very concerned with being able to accurately diagnose cancer and to determine the best course of treatment.

Spectroscopic imaging combines digital imaging and molecular spectroscopy techniques, which can include Raman scattering, fluorescence, photoluminescence, ultraviolet, visible, short wave infrared (SWIR), and infrared absorption spectroscopies. When applied to the chemical analysis of materials, spectroscopic imaging is commonly referred to as chemical imaging. Chemical imaging is a reagentless tissue imaging approach based on the interaction of laser light with tissue samples. The approach yields an image of a sample wherein each pixel of the image is the spectrum of the sample at the corresponding location. The spectrum carries information about the local chemical environment of the sample at each location. Instruments for performing spectroscopic (i.e. chemical) imaging typically comprise an illumination source, image gathering optics, focal plane array imaging detectors and imaging spectrometers.

In general, the sample size determines the choice of image gathering optic. For example, a microscope is typically employed for the analysis of sub micron to millimeter spatial dimension samples. For larger objects, in the range of millimeter to meter dimensions, macro lens optics are appropriate. For samples located within relatively inaccessible environments, flexible fiberscope or rigid borescopes can be employed. For very large scale objects, such as planetary objects, telescopes are appropriate image gathering optics.

For detection of images formed by the various optical systems, two-dimensional, imaging focal plane array (FPA) detectors are typically employed. The choice of FPA detector is governed by the spectroscopic technique employed to characterize the sample of interest. For example, silicon (Si) charge-coupled device (CCD) detectors or CMOS detectors are typically employed with visible wavelength fluorescence and Raman spectroscopic imaging systems, while indium gallium arsenide (InGaAs) FPA detectors are typically employed with near-infrared spectroscopic imaging systems.

Spectroscopic imaging of a sample can be implemented by one of two methods. First, a point-source illumination can be provided on the sample to measure the spectra at each point of the illuminated area. Second, spectra can be collected over an entire area encompassing the sample simultaneously using an electronically tunable optical imaging filter such as an acousto-optic tunable filter (AOTF), a multi-conjugate tunable filter (MCF), or a liquid crystal tunable filter (LCTF). Here, the organic material in such optical filters are actively aligned by applied voltages to produce the desired bandpass and transmission function. The spectra obtained for each pixel of such an image thereby forms a complex data set referred to as a hyperspectral image which contains the intensity values at numerous wavelengths or the wavelength dependence of each pixel element in this image.

The ability to determine a disease state is critical to histological analysis. Such testing often requires obtaining the spectrum of a sample at different wavelengths. Conventional spectroscopic devices operate over a limited range of wavelengths due to the operation ranges of the detectors or tunable filters possible. This enables analysis in the Ultraviolet (UV), visible (VIS), near infrared (NW), short wave infrared (SWIR) mid infrared (MIR) wavelengths and to some overlapping ranges. These correspond to wavelengths of about 180-380 nm (UV), 380-700 nm (VIS), 700-2500 nm (NIR), 850-1700 nm (SWIR) and 2500-25000 nm (MIR).

Diagnosis of cancer is the first critical step to cancer treatment. Included in the diagnosis is the type and grade of cancer and the stage of progression. This information drives treatment selection. When cancer is suspected, a patient will have the tumor removed or biopsied and sent for histopathology analysis. Conventional handling involves the tissue undergoing fixation, staining with dyes, mounting and then examination under a microscope for analysis. Typically, the time taken to prepare the specimen is of the order of one day. The pathologist will view the sample and classify the tissue as malignant or benign based on the shape, color and other cell and tissue characteristics. The result of this manual analysis depends on the choice of stain, the quality of the tissue processing and staining, and ultimately on the quality of education, experience and expertise of the specific pathologist.

The detection and diagnosis of cancer is typically accomplished through the use of optical microscopy. A tissue biopsy is obtained from a patient and that tissue is sectioned and stained. The prepared tissue is then analyzed by a trained pathologist who can differentiate between normal, malignant and benign tissue based on tissue morphology. Because of the tissue preparation required, this process is relatively slow. Moreover, the differentiation made by the pathologist is based on subtle morphological differences between normal, malignant and benign tissue based on tissue morphology. For this reason, there is a need for an imaging device that can rapidly and quantitatively diagnose malignant and benign tissue.

Alternatives to traditional surgical biopsy include fine needle aspiration cytology and needle biopsy. These non-surgical techniques are becoming more prevalent as cancer diagnostic techniques because they are less invasive than biopsy techniques that harvest relatively large tissue masses. Fine needle aspiration cytology has the advantage of being a rapid, minimally invasive, non-surgical technique that retrieves isolated cells that are often adequate for evaluation of disease state. However, in fine needle biopsies intact tissue morphology is disrupted often leaving only cellular structure for analysis which is often less revealing of disease state. In contrast, needle biopsies use a much larger gauge needle which retrieve intact tissue samples that are better suited to morphology analysis. However, needle biopsies necessitate an outpatient surgical procedure and the resulting needle core sample must be embedded or frozen prior to analysis.

It is widely recognized among the cancer research community, that there is a need to develop new tools to characterize normal, precancerous, cancerous, and metastatic cells and tissues at a molecular level. These tools are needed to help expand our understanding of the biological basis of cancers. Molecular analysis of tissue changes in cancer improve the quality and effectiveness of cancer detection and diagnosis strategies. The knowledge gained through such molecular analyses helps identify new targets for therapeutic and preventative agents.

Various types of spectroscopy and imaging may be explored for detection of various types of diseases in particular cancers. For example, Raman chemical imaging (RCI) has a spatial resolving power of approximately 250 nm and can potentially provide qualitative and quantitative image information based on molecular composition and morphology. Raman spectroscopy is based on irradiation of a sample and detection of scattered radiation, and it can be employed non-invasively to analyze biological samples in situ. Thus, little or no sample preparation is required. Raman spectroscopy techniques can be readily performed in aqueous environments because water exhibits very little, but predictable, Raman scattering. It is particularly amenable to in vivo measurements as the powers and excitation wavelengths used are non-destructive to the tissue and have a relatively large penetration depth.

The vast majority of diseases, in particular cancer cases, are pathologically diagnosed using tissue from a biopsy specimen. Therefore it is desirable to devise systems and methodologies that use spectroscopic techniques to diagnose biological samples. It is also desirable to devise methodologies that use spectroscopic techniques to differentiate various cell types (e.g., normal, malignant, benign, etc.), to classify biological samples under investigation (e.g., a normal tissue, a diseased tissue, invasive ductal carcinoma disease state and invasive lobular carcinoma disease state), and to also predict clinical outcome (e.g., progressive or non-progressive state of cancer, etc.) of a diseased cell or tissue.

It would be advantageous if a system and method could be devised that would combine the recognizable features of staining and the accuracy and nondesctructablilty of Raman techniques.

### SUMMARY OF THE INVENTION

The present disclosure relates generally to systems and methods for analyzing biological samples. More specifically, the present disclosure provides for diagnosing a disease state of a lung cancer sample using Raman spectroscopic and imaging techniques. A system and method are disclosed herein for determining the diagnosis of a lung neoplasia based on the use of Raman chemical imaging. Raman scattered light measurements may be transformed into a virtual stain that can be fused with other modes of digital imagery to yield a fused image. This image will comprise those visual features associated with traditional staining methods and be recognizable to pathologists. These images may be used by a pathologist to diagnose samples.

A system and method as contemplated herein may further comprise a procedure or algorithm for diagnosis using at least one biological sample and a method of chemometric analysis. The method may also comprise application of a method or algorithm based on measurements of Raman scattered light to an unknown sample resulting in the classification of the sample into a specific lung neoplasia group. These groups may comprise mesothelioma and/or adenocarcinoma; and, more specifically, epithelioid mesothelioma (EM) and metastatic-to-pleura bronchogenic adenocarcinoma (MAC).

The invention of the present disclosure overcomes the limitations of the prior art by providing for a nondestructible means for diagnosing a biological sample while simultaneously providing the recognizable visual features associated with traditional staining methods. By combining digital staining with spectroscopic information, the invention of the present disclosure provides more accurate and reliable diagnostic information in a medium familiar to pathologists.

### BRIEF DESCRIPTION OF THE DRAWINGS

The accompanying drawings, which are included to provide further understanding of the disclosure and are incorporated in an constitute a part of this specification illustrate embodiments of the disclosure, and together with the description, serve to explain the principles of the disclosure.

FIG. 1 is illustrative of a system of the present disclosure.

FIG. 2 is illustrative of a system of the present disclosure.

FIG. 3 is illustrative of a system of the present disclosure.

FIG. 4 is representative of a method of the present disclosure.

FIG. 5 is representative of a method of the present disclosure.

FIG. 6 depicts mean spectra representative of adenocarcinoma and mesothelioma.

FIG. 7 depicts a scatter plot generated by performing PCA.

FIG. 8 depicts a cross validation plot using values obtained from PC2.

FIG. 9 is illustrative of the digital staining capabilities of the present disclosure.

FIG. 10 is illustrative of the digital staining capabilities of the present disclosure.

### DETAILED DESCRIPTION

Reference will now be made in detail to the preferred embodiments of the present disclosure, examples of which are illustrated in the accompanying drawings. Wherever possible, the same reference numbers will be used throughout the specification to refer to same or like parts.

The patent or application file contains at least one drawing executed in color. Copies of this, patent or patent application publication with color drawing(s) will be provided by the Office upon request and payment of the necessary fee.

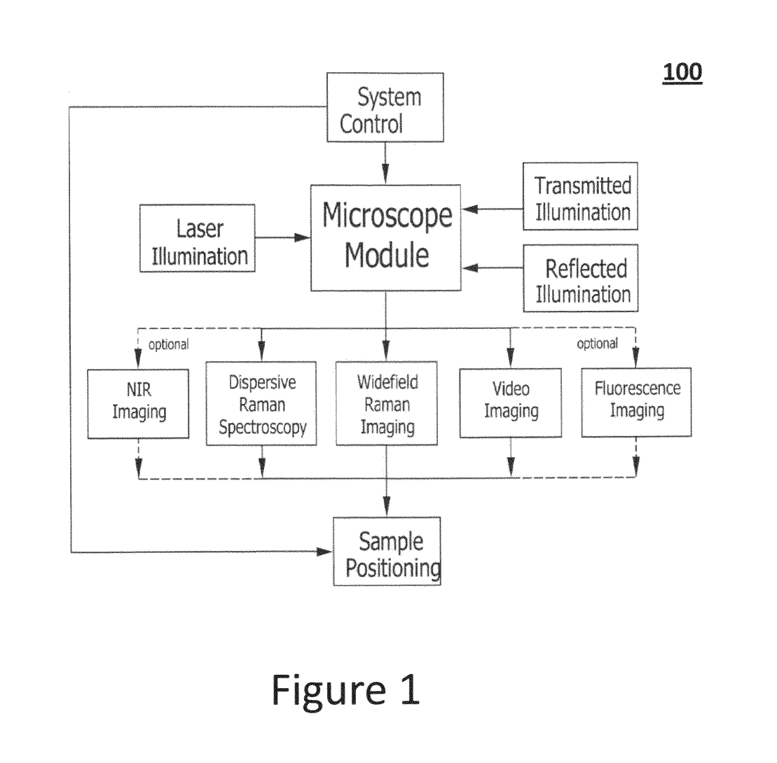

The present disclosure provides for a system and method for diagnosing biological samples that combines the visual staining features familiar to pathologists with the accurate, reliable, and nondestructive capabilities of Raman chemical imaging. FIG. 1 illustrates an exemplary schematic layout of a system of the present disclosure. The layout in FIG. 1 may relate to a chemical imaging system marketed by ChemImage Corporation of Pittsburgh, Pa. In one embodiment, the spectroscopy module 110 may include a microscope module 140 containing optics for microscope applications. An illumination source 142 (e.g., a laser illumination source) may provide illuminating photons to a sample (not shown) handled by a sample positioning unit 144 via the microscope module 140. In one embodiment, photons transmitted, reflected, emitted, or scattered from the illuminated sample (not shown) may pass through the microscope module (as illustrated by exemplary blocks 146, 148 in FIG. 1) before being directed to one or more of spectroscopy or imaging optics in the spectroscopy module 110. The system of FIG. 1 may be configured so as to generate at least one test Raman data set representative of a biological sample under analysis. In the embodiment of FIG. 1, dispersive Raman spectroscopy 152, widefield Raman imaging 154 and video imaging 156 are illustrated as standard. In other embodiments, the modes of NIR imaging 150 and fluorescence imaging 158 may also be implemented.

The spectroscopy module 110 may also include a control unit 160 to control operational aspects (e.g., focusing, sample placement, laser beam transmission, etc.) of various system components including, for example, the microscope module 140 and the sample positioning unit 144 as illustrated in FIG. 1. In one embodiment, operation of various components (including the control unit 160) in the spectroscopy module 110 may be fully automated or partially automated, under user control.

It is noted here that in the discussion herein the terms “illumination,” “illuminating,” “irradiation,” and “excitation” are used interchangeably as can be evident from the context. For example, the terms “illumination source,” “light source,” and “excitation source” are used interchangeably. Similarly, the terms “illuminating photons” and “excitation photons” are also used interchangeably. Furthermore, although the discussion hereinbelow focuses more on Raman spectroscopy and imaging, various methodologies discussed herein may be adapted to be used in conjunction with other types of spectroscopy applications as can be evident to one skilled in the art based on the discussion provided herein.

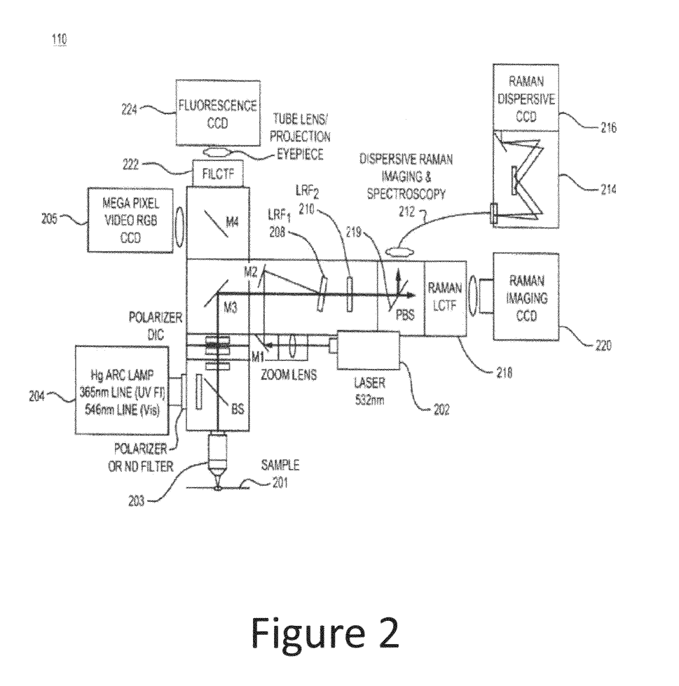

FIG. 2 illustrates exemplary details of the spectroscopy module 110 in FIG. 1 according to one embodiment of the present disclosure. Spectroscopy module 110 may operate in several experimental modes of operation including bright field reflectance and transmission imaging, polarized light imaging, differential interference contrast (DIC) imaging, UV induced autofluorescence imaging, NIR imaging, wide field illumination whole field Raman spectroscopy, wide field spectral fluorescence imaging, wide field visible imaging, wide field SWIR imaging, wide field visible imaging, and wide field spectral Raman imaging. Module 110 may include collection optics 203, light sources 202 and 204, and a plurality of spectral information processing devices including, for example: a tunable fluorescence filter 222, a tunable Raman filter 218, a dispersive spectrometer 214, a plurality of detectors including a fluorescence detector 224, and Raman detectors 216 and 220, a fiber array spectral translator (“FAST”) device 212, filters 208 and 210, and a polarized beam splitter (PBS) 219. At least one Raman detector 216 and 220 may be configured so as to generate at least one test Raman data set representative of a biological sample under analysis.

In one embodiment, a tunable filter may be selected from the group consisting of: a Fabry Perot angle tuned filter, an acousto-optic tunable filter, a liquid crystal tunable filter, a Lyot filter, an Evans split element liquid crystal tunable filter, a Solc liquid crystal tunable filter, a spectral diversity filter, a photonic crystal filter, a fixed wavelength Fabry Perot tunable filter, an air-tuned Fabry Perot tunable filter, a mechanically-tuned Fabry Perot tunable filter, a liquid crystal Fabry Perot tunable filter, and a multi-conjugate tunable filter, and combinations thereof.

In one embodiment, a system of the present disclosure may comprise filter technology available from ChemImage Corporation, Pittsburgh, Pa. This technology is more fully described in the following U.S. Patents and Patent Applications: U.S. Pat. No. 6,992,809, filed on Jan. 31, 2006, entitled “Multi-Conjugate Liquid Crystal Tunable Filter,” U.S. Pat. No. 7,362,489, filed on Apr. 22, 2008, entitled “Multi-Conjugate Liquid Crystal Tunable Filter,” Ser. No. 13/066,428, filed on Apr. 14, 2011, entitled “Short wave infrared multi-conjugate liquid crystal tunable filter.” These patents and patent applications are hereby incorporated by reference in their entireties.

A FAST device may comprise a two-dimensional array of optical fibers drawn into a one-dimensional fiber stack so as to effectively convert a two-dimensional field of view into a curvilinear field of view, and wherein said two-dimensional array of optical fibers is configured to receive said photons and transfer said photons out of said fiber array spectral translator device and to at least one of: a spectrometer, a filter, a detector, and combinations thereof.

The FAST device can provide faster real-time analysis for rapid detection, classification, identification, and visualization of, for example, explosive materials, hazardous agents, biological warfare agents, chemical warfare agents, and pathogenic microorganisms, as well as non-threatening objects, elements, and compounds. FAST technology can acquire a few to thousands of full spectral range, spatially resolved spectra simultaneously, This may be done by focusing a spectroscopic image onto a two-dimensional array of optical fibers that are drawn into a one-dimensional distal array with, for example, serpentine ordering. The one-dimensional fiber stack may be coupled to an imaging spectrometer, a detector, a filter, and combinations thereof. Software may be used to extract the spectral/spatial information that is embedded in a single CCD image frame.

One of the fundamental advantages of this method over other spectroscopic methods is speed of analysis. A complete spectroscopic imaging data set can be acquired in the amount of time it takes to generate a single spectrum from a given material. FAST can be implemented with multiple detectors. Color-coded FAST spectroscopic images can be superimposed on other high-spatial resolution gray-scale images to provide significant insight into the morphology and chemistry of the sample.

The FAST system allows for massively parallel acquisition of full-spectral images. A FAST fiber bundle may feed optical information from is two-dimensional non-linear imaging end (which can be in any non-linear configuration, e.g., circular, square, rectangular, etc.) to its one-dimensional linear distal end. The distal end feeds the optical information into associated detector rows. The detector may be a CCD detector having a fixed number of rows with each row having a predetermined number of pixels. For example, in a 1024-width square detector, there will be 1024 pixels (related to, for example, 1024 spectral wavelengths) per each of the 1024 rows.

The construction of the FAST array requires knowledge of the position of each fiber at both the imaging end and the distal end of the array. Each fiber collects light from a fixed position in the two-dimensional array (imaging end) and transmits this light onto a fixed position on the detector (through that fiber's distal end).

Each fiber may span more than one detector row, allowing higher resolution than one pixel per fiber in the reconstructed image. In fact, this super-resolution, combined with interpolation between fiber pixels (i.e., pixels in the detector associated with the respective fiber), achieves much higher spatial resolution than is otherwise possible. Thus, spatial calibration may involve not only the knowledge of fiber geometry (i.e., fiber correspondence) at the imaging end and the distal end, but also the knowledge of which detector rows are associated with a given fiber.

In one embodiment, a system of the present disclosure may comprise FAST technology available from ChemImage Corporation, Pittsburgh, Pa. This technology is more fully described in the following U.S. Patents, hereby incorporated by reference in their entireties: U.S. Pat. No. 7,764,371, filed on Feb. 15, 2007, entitled “System And Method For Super Resolution Of A Sample In A Fiber Array Spectral Translator System”; U.S. Pat. No. 7,440,096, filed on Mar. 3, 2006, entitled “Method And Apparatus For Compact Spectrometer For Fiber Array Spectral Translator”; U.S. Pat. No. 7,474,395, filed on Feb. 13, 2007, entitled “System And Method For Image Reconstruction In A Fiber Array Spectral Translator System”; and U.S. Pat. No. 7,480,033, filed on Feb. 9, 2006, entitled “System And Method For The Deposition, Detection And Identification Of Threat Agents Using A Fiber Array Spectral Translator”.

In one embodiment, a processor may be operatively coupled to light sources 202 and 204, and the plurality of spectral information processing devices 214, 218 and 222. In another embodiment, a processor, when suitably programmed, can configure various functional parts of a system and may also control their operation at run time. The processor, when suitably programmed, may also facilitate various remote data transfer and analysis operations. Module 110 may optionally include a video camera 205 for video imaging applications. Although not shown in FIG. 2, spectroscopy module 110 may include many additional optical and electrical components to carry out various spectroscopy and imaging applications supported thereby.

A sample 201 may be placed at a focusing location (e.g., by using the sample positioning unit 144 in FIG. 1) to receive illuminating photons and to also provide reflected, emitted, scattered, or transmitted photons from the sample 201 to the collection optics 203. Sample 201 may include a variety of biological samples. In one embodiment, the sample 201 includes at least one cell or a tissue containing a plurality of cells. The sample may contain normal (non-diseased or benign) cells, diseased cells (e.g., cancerous tissues with or without a progressive cancer state or malignant cells with or without a progressive cancer state) or a combination of normal and diseased cells. In one embodiment, the cell/tissue is a mammalian cell/tissue. Some examples of biological samples may include prostate cells, kidney cells, lung cells, colon cells, bone marrow cells, brain cells, red blood cells, and cardiac muscle cells. In one embodiment, the biological sample may include lung cells. In another embodiment, the sample 201 may include cells of plants, non-mammalian animals, fungi, protists, and monera. In yet another embodiment, the sample 201 may include a test sample (e.g., a biological sample under test to determine its metabolic state or its disease status or to determine whether it is cancerous state would progress to the next level). The “test sample,” “target sample,” “biological sample,” or unknown sample are used interchangeably herein to refer to a sample or lung sample under investigation, wherein such interchange use may be without reference to such biological sample's metabolic state or disease status.

In one embodiment, a system of the present disclosure may further comprise a reference database comprising at least one reference data set. In such an embodiment, each reference data set in said reference database may be associated with a known disease state. This known disease state may comprise at least one of: adenocarcinoma, mesothelioma, and combinations thereof. In one embodiment, at least one reference data set may comprise at least one of: a reference hyperspectral Raman image, a reference Raman spectrum, a reference Raman chemical image, and combinations thereof. In one embodiment, said reference data set may comprise a plurality of reference Raman spectra obtained from one or more regions of interest of a known sample.

In one embodiment, a system of the present disclosure may comprise a processor configured so as to execute machine readable program code so as to compare said test Raman data set to at least one of said reference data sets to thereby determine a disease state of a sample.

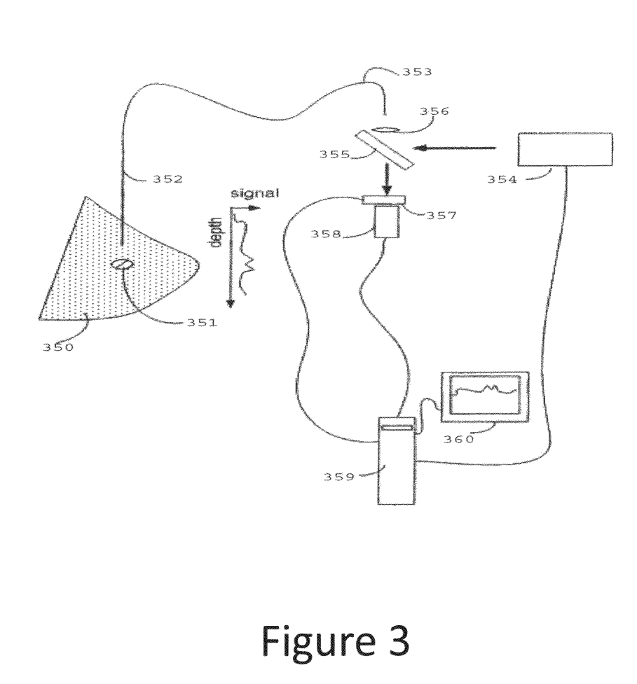

An in vivo embodiment of the invention for examining a lung 350 or other soft tissue for a lesion 351 is shown in FIG. 3. An endoscope or other instrument 352 is used to introduce light carried by an optical fiber 353 from a monochromatic light source 354. A dichroic mirror 355 and lens 356 are shown schematically for introducing the light into the fiber 353. Raman light from the lung is carried from the lung tissue back through the lens 356 and mirror 355, through a filter 357 to a detector 358. The signal from the detector 358 is analyzed by a computer system 359 and displayed on a monitor 360.

The endoscope 352 may comprise an imaging endoscope or fiberscope, where light is conducted from the lung tissue to the detector 358 in a coherent manner through a large plurality of optical fibers. A series of two dimensional images is preferably taken as a function of depth into the tissue and of the Raman shifted wavelength.

Results of an embodiment of the invention is shown by an insert in FIG. 3, where the signal shown is a signal of a molecule indicative of a border region between the lung 350 or other soft tissue and the lesion 351. The spatially resolved signal of tissue or of, for example, carotenoid molecules, is shown in the insert as a function of depth into the lung as the needle carrying the optical fiber is moved into the lung. The signal is shown displayed on the display device 360. In this embodiment, a much finer needle is used than the needle carrying an imaging endoscope. In the fine needle embodiment, the location of the lesion may be more accurately determined, so that fine needle aspiration cytology and/or needle core biopsy may be performed. In the fine needle embodiment, the filter 357 may be a normal spectrometer or a liquid crystal tunable filter.

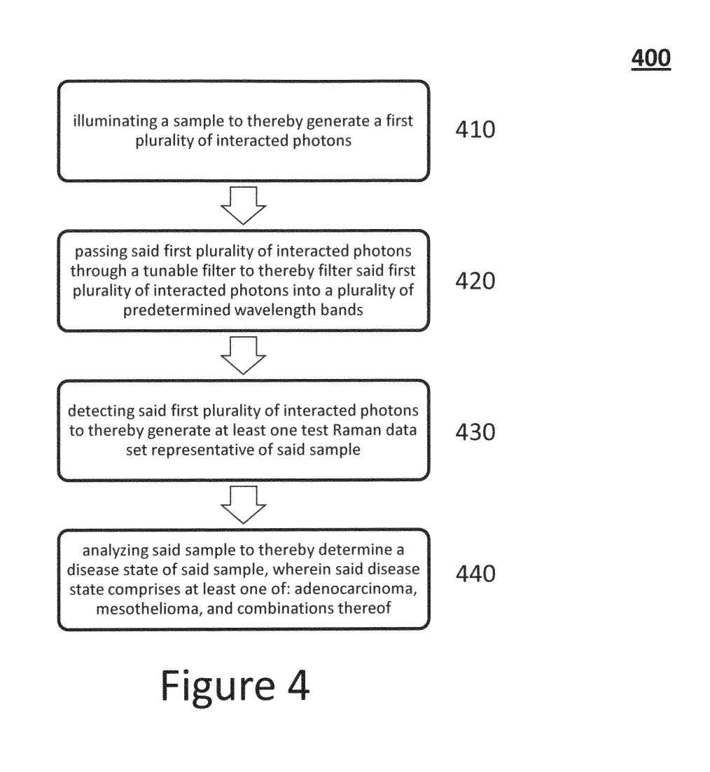

The present disclosure also provides for a method for analyzing a biological sample. In one embodiment, illustrated by FIG. 4, the method 400 may comprise illuminating a sample in step 410 to thereby generate a first plurality of interacted photons. The first plurality of interacted photons may be passes through a tunable filter in step 420 to thereby filter said first plurality of interacted photons into a plurality of predetermined wavelength bands. In step 430 a first plurality of interacted photons may be detected to thereby generate at least one test Raman data set representative of said sample. This test Raman data set may be analyzed in step 440 to thereby determine a disease state of said sample. In the embodiment of FIG. 4, this disease state may comprise at least one of: adenocarcinoma, mesothelioma, and combinations thereof. In one embodiment, the method 400 may further comprise applying at least one digital stain to said test Raman data set.

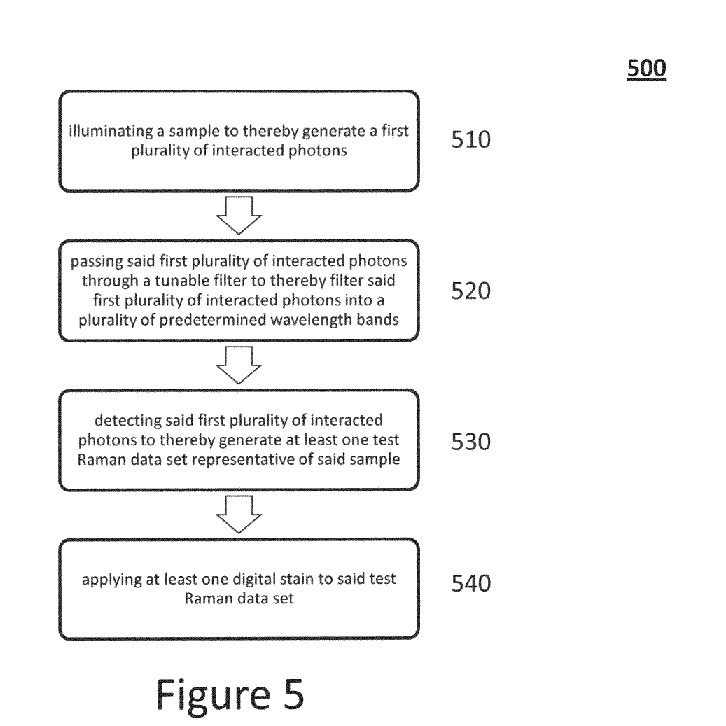

Another embodiment of the present disclosure is illustrated in FIG. 5. In such an embodiment, the method 500 may comprise illuminating a sample in step 510 to thereby generate a first plurality of interacted photons. The first plurality of interacted photons may be passed through a tunable filter in step 520 to thereby filter said first plurality of interacted photons into a plurality of predetermined wavelength bands. In step 530 a first plurality of interacted photons may be detected to thereby generate at least one test Raman data set representative of said sample. In step 540, a digital stain may be applied to said test Raman data set. In one embodiment, the method 500 may further comprise analyzing said test Raman data set to thereby determine a disease state of said sample. This disease state may comprise at least one of: adenocarcinoma, mesothelioma, and combinations thereof.

In one embodiment, the sample under analysis may comprise at least one of: a tissue sample, a cellular sample, and combinations thereof. In one embodiment, the sample may be excised from a patient. Such tissues and cells may be removed from the body in the form of a biopsy, surgical excision, pleural fluid sampling bronchial lavage or other methods established for the extraction of cells and tissues from a patient. In another embodiment, a sample may be analyzed in vivo using a device such as a fiberscope, endoscope, or other suitable device. Such a devise may comprise a rigid or flexible fiberoptic based system that supports Raman scattered light collection and detection. Such a system can be used intra operatively or as part of a diagnostic procedure to provide more information to support diagnostic efforts by surgeons, pathologists, or other medical professionals.

In one embodiment, a test Raman data set may comprise at least one hyperspectral image representative of said sample. In another embodiment, a test Raman data set may comprise at least one of: a Raman chemical image, a Raman spectrum, and combinations thereof. In another embodiment, a test Raman data set may comprise a plurality of Raman spectra obtained from one or more regions of interest of a sample.

In one embodiment, a method of the present disclosure may further comprise analyzing said test Raman data set by comparing said test Raman data set to at least one reference data set in a reference database. Each reference data set may be associated with a known disease state. In one embodiment, this comparison may be achieved by applying at least one chemometric technique. This technique may be selected from the group consisting of: principle component analysis, linear discriminant analysis, partial least squares discriminant analysis, maximum noise fraction, blind source separation, band target entropy minimization, cosine correlation analysis, classical least squares, cluster size insensitive fuzzy-c mean, directed agglomeration clustering, direct classical least squares, fuzzy-c mean, fast non negative least squares, independent component analysis, iterative target transformation factor analysis, k-means, key-set factor analysis, multivariate curve resolution alternating least squares, multilayer feed forward artificial neural network, multilayer perception-artificial neural network, positive matrix factorization, self modeling curve resolution, support vector machine, window evolving factor analysis, and orthogonal projection analysis.

In one embodiment, the present disclosure contemplates that the chemometric technique may be spectral unmixing. The application of spectral unmixing to determine the identity of components of a mixture is described in U.S. Pat. No. 7,072,770, entitled “Method for Identifying Components of a Mixture via Spectral Analysis, issued on Jul. 4, 2006, which is incorporated herein by reference in it entirety. Spectral unmixing as described in the above referenced patent can be applied as follows: Spectral unmixing requires a library of spectra which include possible components of the test sample. The library can in principle be in the form of a single spectrum for each component, a set of spectra for each component, a single Raman image for each component, a set of Raman images for each component, or any of the above as recorded after a dimension reduction procedure such as Principle Component Analysis. In the methods discussed herein, the library used as the basis for application of spectral unmixing is the reference data sets.

With this as the library, a set of measurements made on a sample of unknown state, described herein as a test Raman data set, is assessed using the methods of U.S. Pat. No. 7,072,770 to determine the most likely groups of components which are present in the sample. In this instance the components are actually disease states of interest and/or clinical outcome. The result is a set of disease state groups and/or clinical outcome groups with a ranking of which are most likely to be represented by the test data set.

Given a set of reference spectra, such as those described above, a piece or set of test data can be evaluated by a process called spectral mixture resolution. In this process, the test spectrum is approximated with a linear combination of reference spectra with a goal of minimizing the deviation of the approximation from the test spectrum. This process results in a set of relative weights for the reference spectra.

In one embodiment, the chemometric technique may be Principal Component Analysis. Using Principal Component Analysis results in a set of mathematical vectors defined based on established methods used in multivariate analysis. The vectors form an orthogonal basis, meaning that they are linearly independent vectors. The vectors are determined based on a set of input data by first choosing a vector which describes the most variance within the input data. This first “principal component” or PC is subtracted from each of the members of the input set. The input set after this subtraction is then evaluated in the same fashion (a vector describing the most variance in this set is determined and subtracted) to yield a second vector—the second principal component. The process is iterated until either a chosen number of linearly independent vectors (PCs) are determined, or a chosen amount of the variance within the input data is accounted for.

In one embodiment, the Principal Component Analysis may include a series of steps. A pre-determined vector space is selected that mathematically describes a plurality of reference data sets. Each reference data set may be associated with a known biological sample having an associated metabolic state. The test Raman data set may be transformed into the pre-determined vector space, and then a distribution of transformed data may be analyzed in the pre-determined vector space to generate a diagnosis.

In another embodiment, the Principal Component Analysis may include a series of steps. A pre-determined vector space is selected that mathematically describes a first plurality of reference data sets associated with a known biological sample having an associated diseased state and a second plurality of reference data sets associated with a known biological sample having an associated non-diseased state. The test data set may be transformed into the pre-determined vector space, and then a distribution of transformed data may be analyzed in the pre-determined vector space to generate a diagnosis.

In still yet another embodiment, the Principal Component Analysis may include a series of steps. A pre-determined vector space may be selected that mathematically describes a first plurality of reference data sets associated with a known diagnosis. The test data set may be transformed into the pre-determined vector space, and then a distribution of transformed data may he analyzed in the pre-determined vector space.

The analysis of the distribution of the transformed data may be performed using a classification scheme. Some examples of the classification scheme may include: Mahalanobis distance, Adaptive subspace detector, Band target entropy method, Neural network, and support vector machine as an incomplete list of classification schemes known to those skilled in the art.

In one such embodiment, the classification scheme is Mahalanobis distance. The Mahalanobis distance is an established measure of the distance between two sets of points in a multidimensional space that takes into account both the distance between the centers of two groups, but also the spread around each centroid. A Mahalanobis distance model of the data is represented by plots of the distribution of the spectra in the principal component space. The Mahalanobis distance calculation is a general approach to calculating the distance between a single point and a group of points. It is useful because rather than taking the simple distance between the single point and the mean of the group of points, Mahalanobis distance takes into account the distribution of the points in space as part of the distance calculation. The Mahalanobis distance is calculated using the distances between the points in all dimensions of the principal component space.

In one such embodiment, once the test data is transformed into the space defined by the predetermined PC vector space, the test data is analyzed relative to the pre-determined vector space. This may be performed by calculating a Mahalanobis distance between the test data set transformed into the pre-determined vector space and the data sets in the pre-determined vector space to generate a diagnosis.

Application of a digital stain to a test Raman data set may, in one embodiment, be achieved using a chemometric technique. In one embodiment, principle component analysis (PCA) may be used to color images based on where each pixel lands in the PC2 space. In one embodiment, a leave one out approach may be used where the case to be colored is left out and PCA is performed on the remaining data. Results may be used as reference spectra for a least squares mixing exercise, which may be performed on each pixel of the left out image. In one embodiment, a resulting score image for PC2 may be scaled to have a range of 1. At least one color frame may be developed from a scaled PC2 image. In one embodiment, two independent color frames may be developed. These color frames may be applied to the data, associating various ranges of values with variations of color. In one embodiment, two or more color frames may be merged and overlaid onto an image with the same field of view. This image may be analyzed by visual inspection by a user to thereby diagnose the sample. Spectroscopic information may also be ascertained by comparison with reference data sets associated with a known diagnosis.

In one embodiment, a test Raman data set with a digital stain may be analyzed by visual inspection by a user. The application of the digital stain is not limited to tissue sections but can be applied to cells which are derived from methods used to extract cellular samples from a patient including, but not limited to bronchial lavage, percutaneous pleural effusion fluid sampling, brohchial biopsy, fine needle aspirate or sputum sample. Different methods can be used to prepare the samples from different sampling techniques for optimal performance of Raman analysis.

In one embodiment, a method of the present disclosure may further comprise obtaining a bright field image representative of a sample and fusing this bright field image with a test Raman data set to thereby generate a fused image representative of a sample. This brightfield image may aid in the assessment of morphological characteristics of a biological sample. These morphological characteristics may comprise size, shape, and color of various components of a sample.

In one embodiment, the present disclosure provides for a storage medium containing machine readable program code, which, when executed by a processor, causes said processor to perform the following: illuminate a sample to thereby generate a first plurality of interacted photons; pass said first plurality of interacted photons through a tunable filter to thereby filter said first plurality of interacted photons into a plurality of predetermined wavelength bands; detect said first plurality of interacted photons to thereby generate at least one test Raman data set representative of said sample; and analyze said test Raman data set to thereby determine a disease state of said sample, wherein said disease state comprises at least one of: adenocarcinoma, mesothelioma, and combinations thereof. In one embodiment, the storage medium, which when executed by a processor, may further cause said processor to apply at least one digital stain to said test Raman data set. In another embodiment, the storage medium, when executed by a processor to analyze said test Raman data set, may further cause said processor to compare said test Raman data set to at least one reference data set.

### Example

The following example demonstrates the detection capabilities of the present disclosure. Ten blinded samples were selected for the investigation five samples were obtained from patients with MAC and five samples were obtained from patients with EM. The samples were prepared following standard histopathology techniques to obtain thin sections on microscope slides for analysis. Regions of interest were circled by a pathologist to assist in targeting areas of interest on the tumor. The Falcon II™ Raman imaging microscope, available from ChemImage Corporation, Pittsburgh, Pa., was employed to obtain a Raman chemical image (RCI) on each tissue section. The data was acquired over period of four weeks and at least one data set was obtained on each sample. For some cases where several regions were annotated on a sample, multiple datasets were collected.

The resultant RCI data was processed to minimize both instrumental artifact using a procedure that employs the NIST 2242 Raman spectroscopy standard reference material and background fluorescence using a low order polynomial fit. The images were manually segmented to obtain spectral signatures from desired regions of interest at each field of view that represent epithelial cells. As a result, multiple spectra were extracted for each sample data set, or patient, and in some cases, multiple regions where applicable. The spectra were truncated to the fingerprint region of the spectrum and were grouped by the individual patient number.

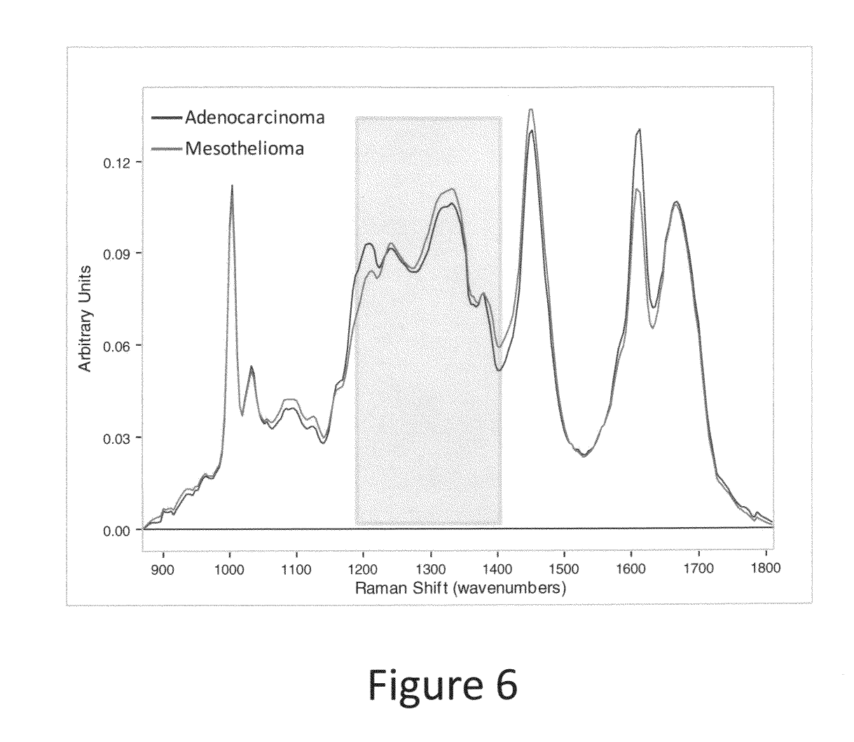

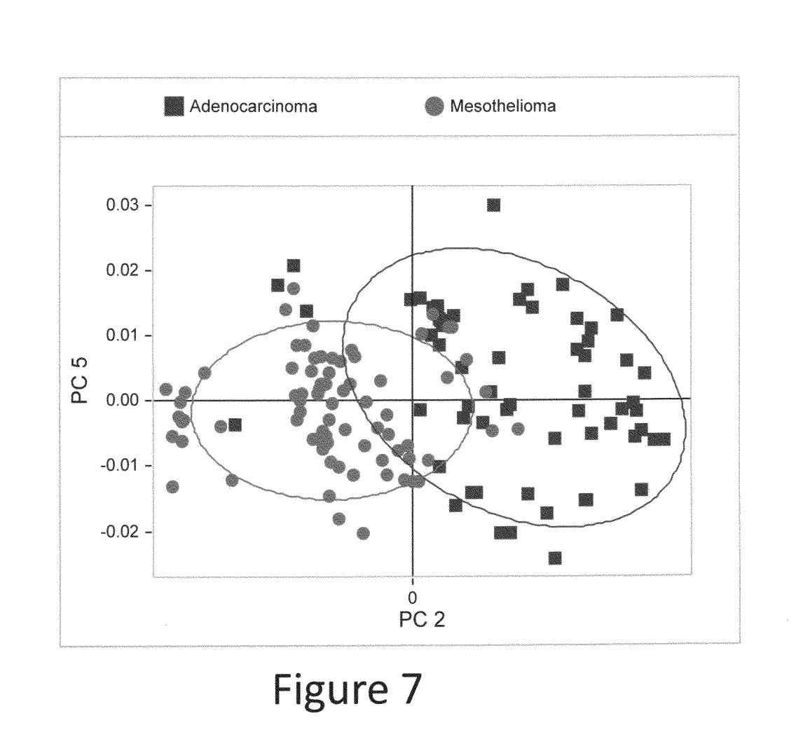

Principal component analysis was applied as an example chemometric method. The present disclosure contemplates that other chemometric techniques applied herein may be applied to the data to determine if the samples separated into two groups based on RCI data. Various new models were created using the unblinded grouping so that an optimized spectral range could be determined. FIG. 6 shows a spectral plot of the mean spectra from the two groups under investigation in this study. By performing PCA on a variety of spectral regions, it was determined that the range of 1200 to 1410 cm⁻¹ yields the largest separation between the groups. The corresponding scatter plot generated from PCA of this region is shown in FIG. 7.

To evaluate performance of this analysis, a leave one out cross validation effort was performed. In the scatter plots shown above, each point represents a spectrum that was extracted from the RCI data. For each case, multiple spectra were extracted from the region measured, or regions measured in some cases. When a leave one out analysis is performed, all of the spectra associated with that patient is left out. The spectra left out are then projected back into the model space to determine where this sample would be located within the two data groups. Various methods may be used to measure and determine where the left out case falls.

In the work presented here, a leave one out analysis was performed using the model generated from the 1200 to 1410 cm⁻¹ spectral region shown in FIG. 7. Each time a sample was left out, the model was regenerated and the left out data was projected onto the plot. The mean PC2 value for each group (MAC and EM) and the left out sample was recorded along with the standard deviation. The process was repeated for each of the ten samples.

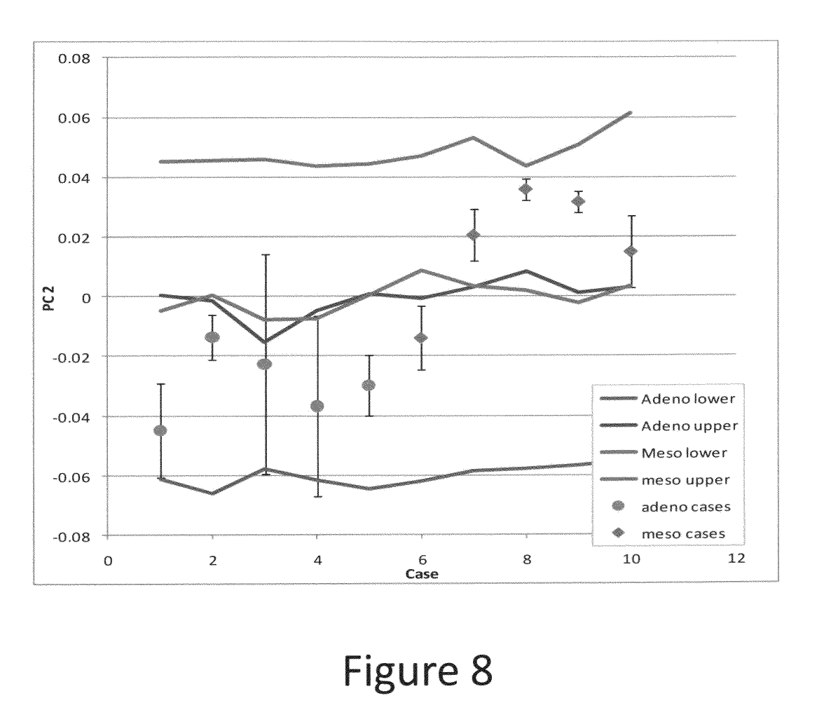

The resultant mean PC2 values recorded can be plotted as a function of case to create a cross validation plot as shown in FIG. 8. In this plot, the solid lines shown are bounds for each group. These bounds are determined using the mean value along PC2 and the standard deviation. With the bounds created for each group, the left out case can be projected onto the scatter plot. The measured mean and standard deviation of PC2 is then used to determine to which group it belongs. These left out cases are shown as solid points in the plot in FIG. 8.

It can be seen that using this method correctly identifies 9 out of the 10 cases correctly. The projected values of performance are listed in table 1.

TABLE 1
Performance of model as a test for adenocarcinoma

| Parameter | From PC2 |
|---|---|
| True Positives | 5 |
| True Negatives | 4 |
| False Positives | 1 |
| False Negatives | 0 |
| Sensitivity | 100% |
| Specificity | 80% |

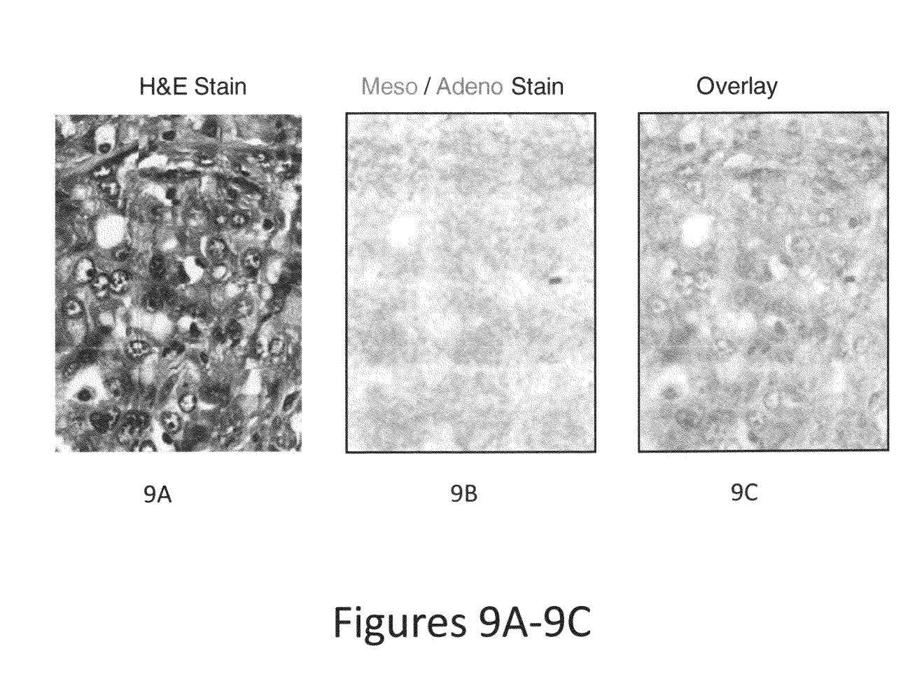

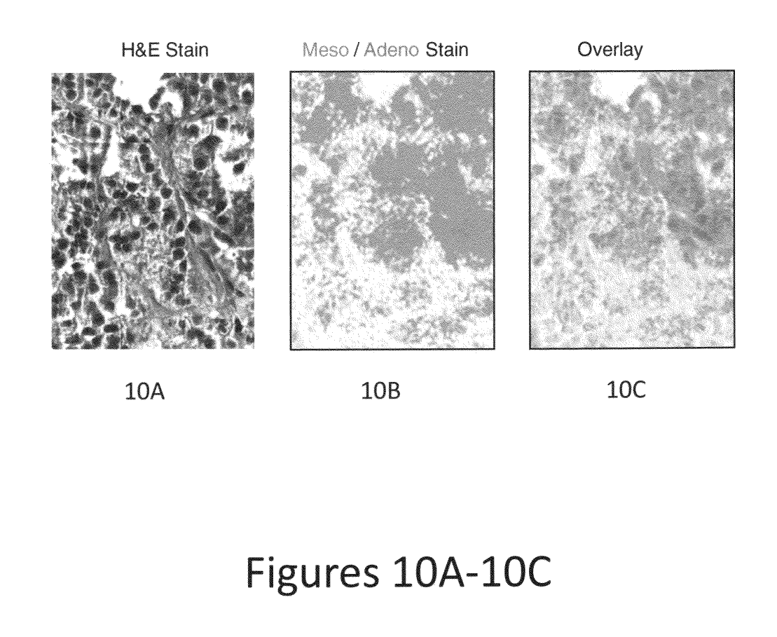

To extend this work from simple PC analysis to actual imaging representation of the RCI information a process was used to color the images based on where each pixel lands in the PC2 space. For this process the leave one out approach was used. The case to be colored was left out and a PCA was performed on the remaining data. The loadings resulting from this exercise were used as library spectra for a least squares unmixing exercise performed on each pixel of the held out image. The resulting score image for PC2 was scaled to have a range of 1. Two independent colored frames were developed from the scaled PC2 score image. The first mapped the values from −0.2 to 0 onto a brown color with −0.2 being the darkest, and 0 being the most faint. Similarly the values from 0 to 0.2 were mapped onto a green color. The brown and green frames were merged and the merged image was overlaid onto an image of the same FOVs after the sample is stained. FIG. 9 shows the result for an example epithelioid mesothelioma case while FIG. 10 shows the result for an example adenocarcinoma case. Other chemometric methods such as those listed above can be used to similarly generate a digital stain.

The Example present here illustrates the potential of the invention of the present disclosure to differentiate between various types of lung cancer and diagnose lung neoplasia based on Raman chemical imaging.

While the disclosure has been described in detail in reference to specific embodiments thereof, it will be apparent to one skilled in the art that various changes and modifications can be made therein without departing from the spirit and scope of the embodiments. Thus, it is intended that the present disclosure cover the modifications and variations of this disclosure provided they come within the scope of the appended claims and their equivalents.

What is claimed is:

1. A method comprising:
illuminating a sample to thereby generate a first plurality of interacted photons;
passing said first plurality of interacted photons through a tunable filter to thereby filter said first plurality of interacted photons into a plurality of predetermined wavelength bands;
detecting said first plurality of interacted photons to thereby generate at least one test Raman data set representative of said sample; and
analyzing said test Raman data set to thereby determine a disease state of said sample, wherein said disease state comprises at least one of: adenocarcinoma, mesothelioma, and combinations thereof.

2. The method of claim 1 wherein said sample comprises at least one of: a tissue sample, a cellular sample, and combinations thereof.

3. The method of claim 1 wherein said sample is excised from a patient.

4. The method of claim 1 wherein said method is performed in vivo.

5. The method of claim 1 wherein said method is performed via an endoscope, a fiberscope, and combinations thereof.

6. The method of claim 1 further comprising obtaining a brightfield image representative of said sample and fusing said brightfield image with said test Raman data set to thereby generate a fused image representative of said sample.

7. The method of claim 1 further comprising applying at least one digital stain to said test Raman data set.

8. The method of claim 1 wherein said test Raman data set comprises a hyperspectral Raman image.

9. The method of claim 1 wherein said test Raman data set comprises at least one of: a Raman chemical image, a Raman spectrum, and combinations thereof.

10. The method of claim 1 wherein said test Raman data set comprises a plurality of Raman spectra obtained from one or more regions of interest of said sample.

11. The method of claim 1 wherein said filtering is further achieved by using a filter selected from the group consisting of: a liquid crystal tunable filter, a multi-conjugate liquid crystal tunable filter, an acousto-optical tunable filter, a Lyot liquid crystal tunable filter, an Evans split-element liquid crystal tunable filter, a Solc liquid crystal tunable filter, a ferroelectric liquid crystal tunable filter, a Fabry Perot liquid crystal tunable filter, and combinations thereof.

12. The method of claim 1 wherein said analyzing further comprises comparing said test Raman data set to at least one reference data set in a reference database, wherein each said reference data set is associated with a known disease state.

13. The method of claim 12 wherein said comparing is achieved by applying at least one chemometric technique.

14. The method of claim 12 wherein said chemometric technique is selected from the group consisting of: principle component analysis, linear discriminant analysis, partial least squares discriminant analysis, maximum noise fraction, blind source separation, band target entropy minimization, cosine correlation analysis, classical least squares, cluster size insensitive fuzzy-c mean, directed agglomeration clustering, direct classical least squares, fuzzy-c mean, fast non negative least squares, independent component analysis, iterative target transformation factor analysis, k-means, key-set factor analysis, multivariate curve resolution alternating least squares, multilayer feed forward artificial neural network, multilayer perception-artificial neural network, positive matrix factorization, self modeling curve resolution, support vector machine, window evolving factor analysis, and orthogonal projection analysis.

15. A method comprising:
illuminating a sample to thereby generate a first plurality of interacted photons;
passing said first plurality of interacted photons through a tunable filter to thereby filter said first plurality of interacted photons into a plurality of predetermined wavelength bands;
detecting said first plurality of interacted photons to thereby generate at least one test Raman data set representative of said sample; and
applying at least one digital stain to said test Raman data set.

16. The method of claim 15 further comprising analyzing said test Raman data set to thereby determine a disease state of said sample, wherein said disease state comprises at least one of: adenocarcinoma, mesothelioma, and combinations thereof.

17. The method of claim 15 wherein said sample comprises at least one of: a tissue sample, a cellular sample, and combinations thereof.

18. The method of claim 15 wherein said sample is excised from a patient.

19. The method of claim 15 wherein said method is performed in vivo.

20. The method of claim 15 wherein said method is performed via an endoscope, a fiberscope, a borescope, and combinations thereof.

21. The method of claim 15 wherein said test Raman data set comprises a hyperspectral Raman image.

22. The method of claim 15 wherein said test Raman data set comprises at least one of: a Raman chemical image, a Raman spectrum, and combinations thereof.

23. The method of claim 15 wherein said test Raman data set comprises a plurality of Raman spectra obtained from one or more regions of interest of said sample.

24. The method of claim 16 wherein said analyzing is achieved by visual inspection of said digital stain by a user.

25. The method of claim 16 wherein said analyzing further comprises comparing said test Raman data set to at least one reference data set in a reference database, wherein each said reference data set is associated with a known disease state.

26. The method of claim 25 wherein said comparing is achieved by applying at least one chemometric technique.

27. The method of claim 26 wherein said chemometric technique is selected from the group consisting of: principle component analysis, linear discriminant analysis, partial least squares discriminant analysis, maximum noise fraction, blind source separation, band target entropy minimization, cosine correlation analysis, classical least squares, cluster size insensitive fuzzy-c mean, directed agglomeration clustering, direct classical least squares, fuzzy-c mean, fast non negative least squares, independent component analysis, iterative target transformation factor analysis, k-means, key-set factor analysis, multivariate curve resolution alternating least squares, multilayer feed forward artificial neural network, multilayer perception-artificial neural network, positive matrix factorization, self modeling curve resolution, support vector machine, window evolving factor analysis, and orthogonal projection analysis.

28. The method of claim 15 wherein said filtering is further achieved by using a filter selected from the group consisting of: a liquid crystal tunable filter, a multi-conjugate liquid crystal tunable filter, an acousto-optical tunable filter, a Lyot liquid crystal tunable filter, an Evans split-element liquid crystal tunable filter, a Solc liquid crystal tunable filter, a ferroelectric liquid crystal tunable filter, a Fabry Perot liquid crystal tunable filter, and combinations thereof.

29. The method of claim 15 further comprising generating a brightfield image representative of said sample and fusing said brightfield image and said test Raman data set to thereby generate a fused image representative of said sample.

30. A system comprising:
a reference database comprising at least one reference data set, wherein each reference data set is associated with a known disease state;
an illumination source configured to illuminate a sample to thereby generate a first plurality of interacted photons;
a tunable filter configured so as to filter said first plurality of interacted photons into a plurality of predetermined wavelength bands;
a detector configured so as to detect said first plurality of interacted photons and thereby generate a test Raman data set representative of said sample;
a machine readable program code containing executable program instructions; and
a processor operatively coupled to the illumination source and the detector, and configured to execute said machine readable program code so as to perform the following:
compare said test Raman data set to at least one of said reference data sets to thereby determine a disease state of said sample, wherein said disease state comprises at least one of: adenocarcinoma, mesothelioma, and combinations thereof.

31. The system of claim 30 wherein said filter is selected from the group consisting of: a liquid crystal tunable filter, a multi-conjugate liquid crystal tunable filter, an acousto-optical tunable filter, a Lyot liquid crystal tunable filter, an Evans split-element liquid crystal tunable filter, a Solc liquid crystal tunable filter, a ferroelectric liquid.

32. The system of claim 30 further comprising at least of: an endoscope, a fiberscope, and combinations thereof.

33. A storage medium containing machine readable program code, which, when executed by a processor, causes said processor to perform the following:
illuminate a sample to thereby generate a first plurality of interacted photons;
pass said first plurality of interacted photons through a tunable filter to thereby filter said first plurality of interacted photons into a plurality of predetermined wavelength bands;
detect said first plurality of interacted photons to thereby generate at least one test Raman data set representative of said sample; and
analyze said test Raman data set to thereby determine a disease state of said sample, wherein said disease state comprises at least one of: adenocarcinoma, mesothelioma, and combinations thereof.

34. The storage medium of claim 33, which when executed by a processor, further causes said processor to apply at least one digital stain to said test Raman data set.

35. The storage medium of claim 33, which when executed by a processor to analyze said test Raman data set, further causes said processor to compare said test Raman data set to at least one reference data set.
